(19) United States
(12) Patent Application Publication
(10) Pub. No.: US 2013/0281877 A1
(43) Pub. Date: Oct. 24, 2013

(54) Skin Temperature Control in Therapeutic Medical Ultrasound

(71) Applicants: Stephen J. Hsu, Issaquah, WA (US); Liexiang Fan, Sammamish, WA (US); Kevin Michael Sekins, Yarrow Point, WA (US)

(72) Inventors: Stephen J. Hsu, Issaquah, WA (US); Liexiang Fan, Sammamish, WA (US); Kevin Michael Sekins, Yarrow Point, WA (US)

(73) Assignee: Siemens Medical Solutions USA, Inc, Malvern, PA (US)

(21) Appl. No.: 13/451,423

(22) Filed: Apr. 19, 2012

(51) Int. Cl. A61B 5/00; A61N 7/00

(52) U.S. Cl. 600/549; 601/2

(57) ABSTRACT

Skin temperature is measured during medical ultrasound therapy. The temperature of a standoff between the transducer and skin is monitored. The temperature of the standoff relates to the skin temperature. The temperature, whether skin or standoff temperature, is used to control the therapy. The temperature feedback may allow for increased or optimize therapy levels.

### BACKGROUND

The present invention relates to ultrasound therapy. For example, high intensity focused ultrasound (HIFU) is applied to a region within a patient for treatment, such as by heating the region.

To heat the region, a transducer generates acoustic energy. The acoustic energy propagates from the transducer, through the skin, and into the patient. While the region is to be heated, other locations, such as the skin, are not to be heated. However, the acoustic energy may heat the skin. Skin heating occurs due to the acoustic impedance mismatch at the point of contact between the body and the transducer or coupling agent between the body and the transducer. The transducer may also heat the skin. Skin heating is a negative side effect of HIFU therapy.

Skin heating may be minimized by planning. The duration, pulse repetition interval, or other characteristic of the acoustic energy may be set to limit skin heating. However, these settings may limit the actual therapy.

### BRIEF SUMMARY

By way of introduction, the preferred embodiments described below include methods, computer readable media, instructions, and systems for determining skin temperature in medical ultrasound therapy. The temperature of a standoff between the transducer and skin is monitored with ultrasound. The temperature of the standoff relates to the skin temperature. The skin temperature is used to control the therapy. The temperature feedback may allow for increased or optimized therapy levels.

In a first aspect, a method is provided for determining skin temperature in medical ultrasound therapy. A standoff is positioned between a therapy transducer and skin of a patient. A thermal dose is applied from the therapy transducer, through the standoff, through the skin, and into the patient. The thermal dose is focused at a region in the patient such that the region is heated in response to the thermal dose. The therapy transducer is used to acquire ultrasound data representing acoustic echoes from the standoff adjacent to the skin. The skin temperature is determined as a function of the acoustic echoes. The application of the thermal dose is controlled as a function of the skin temperature.

In a second aspect, a non-transitory computer readable storage medium has stored therein data representing instructions executable by a programmed processor for determining skin temperature in medical ultrasound therapy. The storage medium includes instructions for performing a high intensity focused ultrasound (HIFU) procedure with a HIFU applicator, monitoring a phantom temperature of a phantom material between the HIFU applicator and a patient, relating the phantom temperature to a skin temperature, and adjusting the HIFU procedure based on the skin temperature.

In a third aspect, a system is provided for controlling skin temperature in medical ultrasound therapy. A pad is operable to allow propagation of acoustic energy from an array of acoustic elements and to reflect the acoustic energy to the array. A receive beamformer is configured to acquire ultrasound data representing the pad. A processor is configured to control generating of heat in a patient as a function of the ultrasound data representing the pad.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the Figures, like reference numerals designate corresponding parts throughout the different views.

FIG. 1 is a flow chart diagram of one embodiment of a method for controlling skin temperature in medical ultrasound therapy;

FIG. 2 illustrates an arrangement for monitoring skin temperature while treating a region, according to one embodiment;

FIG. 3 is an example of a standoff; and

FIG. 4 is a block diagram of one embodiment of a system for controlling skin temperature in medical ultrasound therapy.

### DETAILED DESCRIPTION OF THE DRAWINGS AND PRESENTLY

### Preferred Embodiments

A high intensity focused ultrasound (HIFU) system monitors skin temperatures and adjusts the therapy procedure accordingly. A layer of phantom material is placed between the therapy device and body of the patient. Absolute temperature or changes in temperature within this intermediate layer may be characterized by ultrasound imaging. The temperature measured within this layer is taken to reflect the skin temperature. Any adjustments in the HIFU therapy sequence may be determined from the skin temperature.

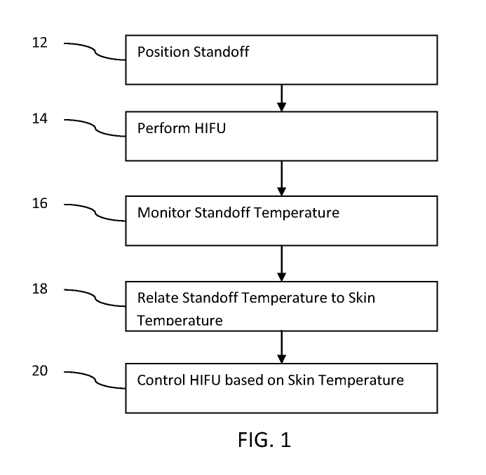

FIG. 1 shows one embodiment of a method of controlling skin temperature in medical ultrasound therapy. This embodiment is directed to control of therapy to control skin temperature. Avoiding overheating the skin while optimizing the thermal dose may be provided. The acts are performed in the order shown or a different order. For example, act 14 is on-going or interleaved with act 16. Additional, different, or fewer acts may be provided. For example, acts 16 and 18 may be collapsed into one act, such as where the temperature of the standoff is used as the skin temperature. As another example, act 20 is not performed, such as where no change is needed as the skin temperature is not a concern. In yet another example, the temperature of the treatment region or other regions within the patient is also monitored with ultrasound. For another example, an image of the skin and/or other temperatures may be generated.

In act 12, a standoff is positioned between a therapy transducer and skin of a patient. Any standoff may be used, such as a gel pad, phantom, block, liquid filled bag, plastic, gelatin, or other material. The standoff spaces the transducer from the skin. Any spacing may be used, such as 1-3 centimeters or about two centimeters or less. About is used to account for variations due to tolerance and/or skin surface irregularities.

The user positions the standoff against the patient. The transducer is then positioned on the standoff. Straps, pressure, glue, gel, or other technique may be used for holding the standoff on the patient. In other embodiments, the standoff is mounted to or otherwise integrated with the transducer probe, so placement of the transducer also places the standoff.

For acoustic impedance matching, gel may be applied. The gel is applied to the skin, standoff, and/or the transducer. For example, gel is applied at the interface of the transducer with the standoff, and gel is applied at the interface of the standoff with the skin of the patient.

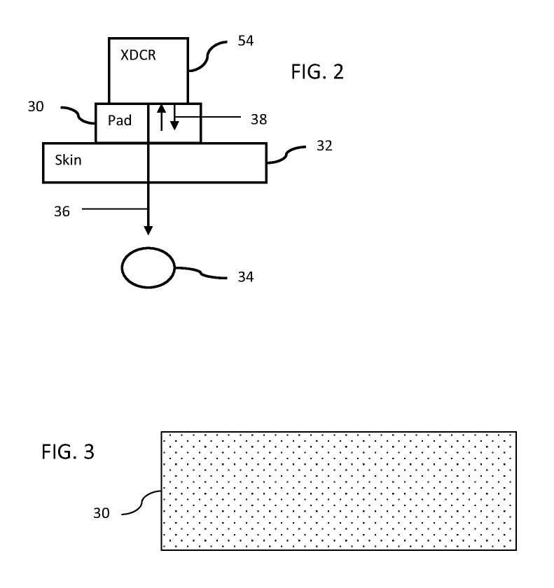

In act 14, a HIFU procedure is performed with a HIFU applicator (e.g., transducer). The transducer generates acoustic energy focused at a treatment region. As shown in FIG. 2, a transducer 54 generates acoustic energy, represented as the ray 36, for treating the region 34 within the patient. The acoustic energy passes through the standoff 30, the skin 32, and into the patient.

To generate the thermal dose at the region 34, the elements of the transducer are driven. Electrical waveforms are applied to the elements. By timing the wavefronts for the different elements, an acoustic beam with a point, line, area, or region focus is generated. Acoustic energy propagates from the various elements of the aperture, and the corresponding wavefronts constructively interfere along the beam and at the focus. The focus is positioned at the region 34 for treatment, but may be positioned elsewhere.

The electrical waveforms are generated by transmitters in the applicator and/or in a separate therapy system. The transmitters operate in response to delays and/or phasing from a transmit beamformer. Apodization control may also be used.

The electrical waveforms for any given therapy beam may be triggered. For interleaving, the generation of therapy beams is controlled to avoid interference with temperature measurements. The trigger may additionally or alternatively be for controlling when all desired arrangements have been made and the patient is ready for treatment.

In response to the electrical waveforms, the therapy beam is generated. Any level of therapy may be applied. For example, an acoustic power greater than 100 watts is transmitted from the transducer to provide high intensity focused ultrasound. The acoustic power causes heating. In response to the thermal dose, the region 34 is heated.

The therapy beam may have various characteristics. The amplitude, aperture size, aperture position, pulse repetition frequency, waveform frequency, duration of application (total number of separate pulses), duration of a given pulse (e.g., number of cycles of the waveform), and/or other characteristics are controlled to provide the desired thermal dose and corresponding treatment. The focus may be shifted over time to treat a larger region.

In act 16, a temperature of the standoff is monitored. The temperature in the region between the transducer 54 and the patient (e.g., skin 32) is measured. The temperature within the layer of standoff material (e.g., layer of phantom material) may be monitored.

The monitoring of temperature occurs during the HIFU procedure. The temperature is measured during therapy. The measurements are interleaved with the therapy. Alternatively, frequency differences or other coding are used to perform both ultrasound temperature measurement and therapy simultaneously. In yet another embodiment, echoes from the therapy waveform are received and used for temperature determination.

The temperature measurement may be repeated throughout the therapy. For example, a reference set of data is acquired before application of the therapy. One or more parameters may be assumed for the initial iteration, such as assuming a temperature common for patients or type of tissue within a patient. Once thermal therapy begins, the temperature measurements are repeated to provide updated measurements. Changes in temperature may be measured. Alternatively, an absolute temperature at any given time is measured.

To measure temperature, the same transducer 54 as used for therapy is used. Ultrasound may be used to measure temperature. The ultrasound transducer scans or images at or by the interface between the phantom material layer (e.g., standoff) and the patient. The ultrasound system scans a region of the standoff at the interface. The interface itself and/or locations adjacent to the interface are scanned. The scan is for one or more locations. For example, data representing a line, in a plane, or in a volume is received with the transducer 54. The focal regions for sampling are set to be within the standoff, such as within the standoff at the interface. Ultrasound data representing the standoff adjacent to the skin is acquired.

Any type of scan, scan format, or imaging mode may be used. For example, harmonic imaging is used. As another example, B-mode, M-mode, or other imaging mode is used. A temperature measurement mode not otherwise used for imaging may be used.

Waveforms at ultrasound frequencies are transmitted, and echoes are received by the HIFU applicator (i.e., the transducer 54). FIG. 2 represents these transmission and reception events with rays 38. The rays 36 and 38 are simplified to represent beam directions or scan lines rather than paths of travel of all the acoustic energy from and to the array.

The standoff includes scatterers. FIG. 3 shows the standoff 30 with particles. The particles are of aluminum or other metal material. Other particles may be used. In other embodiments, the standoff itself includes discontinuities (e.g., air bubbles or imperfections of a sufficient scale to be detected) that reflect some of the acoustic energy. In yet other embodiments, devices, such as wires or spheres, are included in the standoff for reflecting acoustic energy.

The acoustic echoes are converted into electrical signals and beamformed to represent sampled locations within the standoff. The beamformed data may be filtered or otherwise processed. The beamformed data may be detected, such as determining an intensity, or may be radio frequency data prior to any detection (e.g., in-phase and quadrature data). A sequence of echo signals from a same location may be used to measure the temperature. Echoes at one or more harmonics of the transmitted waveforms may be processed. The detected values may be filtered and/or scan converted to a display format. The ultrasound data representing the standoff is from any point along the ultrasound processing path, such as channel data prior to beamformation, radio frequency or in-phase and quadrature data prior to detection, detected data, or scan converted data.

In act 18, the skin temperature is determined. The acoustic echoes from the monitoring are used to find the skin temperature. Any data derived from the acoustic echoes may be used.

The skin temperature is determined from a temperature of the standoff or interface of the standoff with the skin. Any now known or later developed technique for determining temperature may be used. Any temperature related measurement may be used. For example, the standoff material may expand when heated. Measuring the expansion may indicate temperature. The change in distance between two specific reflectors may be measured to indicate expansion. Temperature related measurements may directly or indirectly indicate a temperature. The measurements may be raw ultrasound data or may be derived from ultrasound data.

Measurements are performed for just one location, or for multiple locations in the standoff. Full or sparse sampling may be used. The measurements are performed over time, but independent of previous measurements. Alternatively or additionally, a change in a measurement from a reference or any previous (e.g., most recent) measurement may be used.

In addition to data from acoustic echoes, other information may be used to measure the temperature. Information from the HIFU therapy may be used, such as a thermal dose estimate. An energy output, dose, or other parameter of the thermal treatment is measured or received.

Non-real time measurements may be used, such as a baseline temperature. MRI-based measurements for temperature distribution in a region may be used. Real-time measurements may be used, such as associated with ultrasound measurements performed during application of thermal therapy to a region of the patient.

In one embodiment, one or more ultrasound measurements are performed with or without other temperature related measurements. Any now known or later developed temperature related measurement using ultrasound may be used. In one embodiment, one or more, such as all four, of tissue displacement, speed of sound, backscatter intensity, and a normalized correlation coefficient of received signals are performed. Other measurements are possible, such as expansion.

Displacement is measured by determining an offset in one, two, or three-dimensions. A displacement associated with a minimum sum of absolute differences or highest correlation is determined. The current scan data is translated, rotated, and/or scaled relative to a reference dataset, such as a previous or initial scan. The offset associated with a greatest or sufficient similarity is determined as the displacement. B-mode or harmonic mode data is used, but other data may be used. The displacement calculated for one location may be used to refine the search or search region in another location. Other measures of displacement may be used.

The speed of sound may be measured by comparison from prior to heating with during heating. A pulse is transmitted. The time for the echo to return from a given location may be used to determine the speed of sound from the transducer to the location and back. Any aperture may be used, such as separately measuring for the same locations with different apertures and averaging. In another embodiment, signals are correlated. For example, in-phase and quadrature signals after beamformation are correlated with reference signals. A phase offset between the reference and current signals is determined. The frequency of the transmitted waveform (i.e., ultrasound frequency) is used to convert the phase difference to a time or speed of sound. Other measurements of the speed of sound may be used.

The backscatter intensity may be measured. B-mode or M-mode data indicates backscatter intensity. The intensity or energy of the envelope of the echo signal is determined. This intensity may reflect temperature.

The normalized correlation coefficient of received signals may be measured. Beamformed data prior to detection, such as in-phase and quadrature data, is cross-correlated. In one embodiment, a reference sample or samples are acquired. During treatment, subsequent samples are acquired. For each location, a spatial window, such as three wavelengths in depth, defines the data for correlation. The window defines a length, area or volume. The current data is correlated with the reference data within the window space. The normalized cross-correlation is performed for the data in the window. As new data is acquired, further cross-correlation is performed. The normalized correlation coefficient may indicate temperature.

Any temperature associated acoustic and physical parameters or changes in the parameters may be measured. Other measurements include elasticity, strain, strain rate, motion (e.g., displacement), or reflected power (e.g., backscatter cross-section).

The temperature is determined from the measured parameter. The results of the ultrasound and any other measures are related to temperature in the standoff or at the interface. Experimental relationships between the measures and temperature may be used in a look-up table or incorporated into a function for calculating the temperature. The standoff is characterized before the procedure so that an ultrasonic-based thermometry measurement within the material relates to a temperature. For example, coefficients relating thermal strain to temperature are used.

A temperature is determined for each measurement location. The temperatures may be used separately, such as showing a pattern of temperature. The temperatures may be combined, such as providing an average temperature. In one embodiment, a peak temperature is identified. Alternatively, the temperature is determined in general from measurements of just one location or of multiple locations.

The temperature of the standoff is related to the skin temperature. This relationship may be specifically determined as part of the look-up table or temperature calculation. For example, the measures of the characteristic of the standoff are used to estimate the skin temperature without a separate calculation of the standoff temperature even where the measurements are for locations in the standoff. In one embodiment, a thermal characteristic of the standoff material and the distance of the measurement location from the skin are used to calculating an estimate of the skin temperature from a specifically determined standoff temperature. A linear or non-linear relationship of standoff temperature to skin temperature may be used.

The skin temperature may be assumed to be the same as the standoff temperature. The relationship is one-to-one. For example, the skin temperature is identified based on thermal strain of the standoff material adjacent to an interface of the standoff with skin of the patient. No further derivation is used. This standoff temperature measurement is taken to reflect the skin temperature.

In one embodiment, the relationship is modeled as a thermal distribution model. The standoff temperature at one location may be used to derive the temperature at a different location, such as at the interface. The skin temperature is estimated from a measured characteristic of the standoff with a thermal distribution model. For example, the thermal distribution model is applied to the temperature. The thermal distribution model accounts for the type or types of material of the standoff and relative distribution of materials.

The thermal distribution model may be used to determine temperatures at locations other than the measurement locations. The input information is sparse, such as a temperature in time and/or location less than all times or locations. The thermal distribution model is used to determine the temperature at other times or locations.

In one embodiment, the relationship between the measurements and skin temperature and/or between standoff temperature and the skin temperature is represented by a model. The model is programmed or designed based on theory or experimentation. The received signals representing the acoustic echoes or data derived from the received signals are applied as inputs to the model.

In one embodiment, the model is a machine-trained model. For example, recursive neural network coefficients of various ultrasound-derived features relate the input to temperature. Any model may be used, such as a neural network or a piecewise linear model. Examples of modeling to determine temperature are disclosed in U.S. Published Patent Application No. 2011/0060221, the disclosure of which is incorporated herein by reference. These models without anatomy information or using standoff characteristics as the anatomy information may be used to estimate skin temperature. The model is trained from a set of training data labeled with a ground truth, such as training data associated with actual temperatures. For example, the various measures or receive data are acquired over time for standoffs. During thermal therapy, the temperature is measured. The temperature is the ground truth. Through one or more various machine-learning processes, the model is trained to predict temperature given the values and/or any feedback. The machine trained model represents a probability of temperature related information. This probability is a likelihood for the temperature related information. A range of probabilities associated with different temperatures is output. Alternatively, the temperature with the highest probability is output. In other embodiments, the temperature related information is output without probability information.

As an alternative to machine learning, manually programmed models may be used. The model may be validated using machine training or through experimental verification.

The current measurements or a time history of measurements may be used to model the temperature. The output of the model may be used as an input. The values are applied during the application of thermal therapy. For an initial application of the model, the feedback is replaced with a reference temperature, such as the temperature of the patient or a room temperature. For further application of the model, the previous output is fed back as an input, providing a time-dependent model. The temperature related information output by the model is fed back as a time history of the information, such as temperature at one or more other times. During thermal therapy, the measured or received values are updated (i.e., current values are input for each application of the model), but previous values may also be used. The feedback provides an estimated spatial distribution of temperature or related information in the region at a previous time. The subsequent output of the model is a function of the ultrasound data or other values and a previous output of the modeling. The time-history of the values may be used as inputs, such that the time history and spatial distributions of the temperature-associated and therapeutic effect-related parameters are used as features of the model.

The model outputs a temperature or temperature distribution (i.e., temperature at different locations and/or times) from the input information. The derived temperature may be in any unit, such as degrees Fahrenheit or Celsius. The resolution of the temperature may be at any level, such as outputting temperature as in one of multiple three or other degree ranges. Alternatively, other temperature related information is output, such as a change in temperature, a dose, or an index value.

In act 20, the application of the thermal dose is controlled based on the skin temperature. The HIFU procedure is adjusted as a function of the skin temperature. Various adjustments are possible. The thermal dose may be increased or decreased. For decreasing the thermal dose, the application of HIFU may be terminated. By ceasing the performing of the HIFU procedure, damage to the skin may be minimized or avoided. For example, if the temperature is at or above a threshold level associated with burning or pain, the generation of acoustic energy for treatment may stop to avoid further temperature increase.

The thermal dose may be decreased without ceasing application. For example, the pulse repetition interval is increased (e.g., decrease in frequency of pulses), the amplitude of the acoustic waveforms is decreased, the aperture is reduced, or the frequency of the waveform is increased. Any alteration reducing the thermal dose at the treatment region and/or at the skin surface may be used.

The thermal dose is decreased in response to a threshold. Different thresholds may be used for ceasing and decreasing. For example, 45 degrees Celsius is used as a threshold to decrease thermal dose while continuing treatment. If the skin temperature continues to rise to 52 degrees Celsius, then the treatment is ceased. Multiple thresholds may be used for gradually decreasing based on increasing skin temperature. For example, a first reduction is made at one threshold level and another reduction is made at a higher threshold level.

The thermal dose may be increased. A threshold level may indicate little risk or acceptable skin temperature. As long as the skin temperature remains below a given level, the thermal dose may be increased. This may allow for greater thermal dose or more rapid application of thermal dose.

A peak, maximum, average or other temperature is used for controlling the application of treatment. For example, the temperatures at different locations are determined. The different locations are along the interface or skin surface. The peak is identified without or after low pass filtering.

In other embodiments, the distribution of temperatures is used for adjusting the thermal dose or therapy. For example, an area may be associated with elevated temperatures. The aperture position may be altered so that the therapy beam is focused on the treatment region, but with the source of the acoustic energy being shifted. This may avoid increasing or limit any increase in the skin temperature at the locations of already elevated temperatures.

The temporal profile of the temperature may be used. Instead of or in addition to an absolute temperature threshold, the rate of temperature change or other change characteristic may be used to control the therapy.

Any use may be made of the skin temperature measurement. Based on the temperature, the therapy may be controlled. The control is manual, such as the user selecting adjustments or an end point for thermal therapy based on the temperature. Alternatively, the control is automatic, such as ceasing or varying therapy when a temperature and/or dose are reached.

In one embodiment, the skin temperature is input to a dosimetry model. The skin temperature may be used to control the thermal dose through altering the dose plan. The dosimetry model determines the thermal dose, such as the maximum thermal dose in the treatment region, an average or overall dose, or the thermal dosage for different locations. The thermal dose is determined from an amount of time and temperature, but may be based on other factors. The temperatures at different treatment locations are used to determine the dose at the different locations or an overall dose for the regions, such as an average or total dose. Any now known or later developed dosimetry model may be used, such as Saparetto-Dewey, a dosimetry equation, or cumulative equivalent minutes at a reference temperature. The dosimetry model outputs dose.

The temperature may be displayed. A value, such as a skin temperature, is displayed to the physician and/or patient. A graph of skin temperature as a function of time or along a line may be displayed.

In one embodiment, the temperature is mapped to color and overlaid on a two-dimensional image or a three-dimensional representation. The mapping modulates the color as a function of the skin temperature, such as the shade of red or color between red and yellow being different for different temperatures. The change in temperature may alternatively be mapped to the output color or additionally mapped to brightness or other aspect of the color. The overlay is laid over an image representing the skin, such as overlaid on an optical image or a generic skin surface.

The spatial distribution of the temperature or related information is represented by the overlay of the image. A separate temperature image may be generated. The temperature at different locations is indicated.

The images are provided in real-time or as acquired. Other images may be displayed, such as images associated with the treatment region. Temperatures at and around the treatment region may be displayed. The image shows the resulting temperature distribution, providing an indication of the therapeutic effect registered and overlaid on anatomic information.

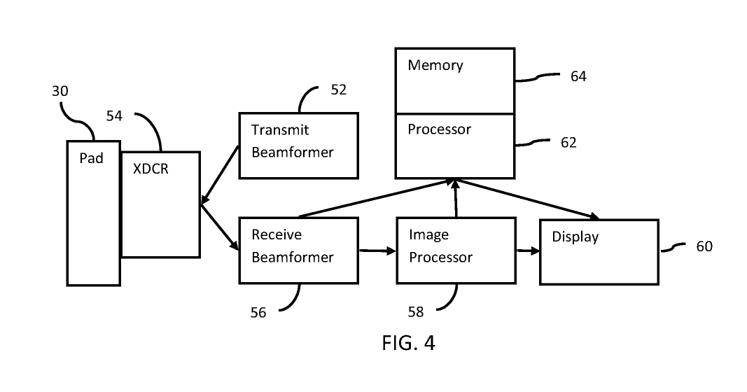

FIG. 4 shows one embodiment of a system for controlling skin temperature in medical ultrasound therapy. The system performs the method described above or a different method. Other systems may be used. The ultrasound system includes a pad 30, a transmit beamformer 52, a transducer 54, a receive beamformer 56, an image processor 58, a display 60, a processor 62 and a memory 64. Additional, different or fewer components may be provided. For example, separate detectors and scan converter are also provided. As another example, a separate therapy transducer or treatment system is provided. In yet another example, the processor 62 and the image processor 58 are combined or one device.

The system 10 is a medical therapeutic ultrasound system. A diagnostic ultrasound imaging system may also be included. Imaging includes two-dimensional, three-dimensional, B-mode, Doppler, color flow, spectral Doppler, M-mode or other imaging modalities now known or later developed. The ultrasound system 10 is a full size cart mounted system, a smaller portable system, a hand-held system or other now known or later developed ultrasound therapy system. In another embodiment, the processor 62 and memory 64 are part of a separate system. For example, the processor 62 and the memory 64 are a workstation or personal computer operating independently of the ultrasound system. As another example, the processor 62 and the memory 64 are part of a diagnostic ultrasound imaging system.

The transducer 54 comprises a single, one-dimensional, multi-dimensional or other now known or later developed ultrasound transducer. For example, the transducer 54 is an array of transducer elements. Each element of the transducer 54 is a piezoelectric, microelectromechanical, capacitive membrane ultrasound transducer, or other now known or later developed transduction element for converting between acoustic and electrical energy. Each of the transducer elements connect to the beamformers 52, 56 for receiving electrical energy from the transmit beamformer 52 and providing electrical energy responsive to acoustic echoes to the receive beamformer 56. The elements may be independently addressable by the beamformers 52, 56.

More than one transducer 54 may be used. For example, the transducer 54 shown in FIGS. 2 and 4 is used for therapy. A separate transducer adjacent to or spaced from the therapy transducer 54 but also in contact with the pad 30 may be used to image or scan the pad 30 or interface with the skin for determining temperature. The separate transducer may scan the pad 30 or interface from a different angle than the therapy transducer 54.

The pad 30 is a standoff, gel pad, pillow, slab, phantom, or other device. The pad 30 has an acoustic impedance similar to water or the transducer 54. In one embodiment, the pad 30 is gelatin, silicone, or other ultrasound phantom material. The pad 30 may include one or more inserts, such as air bubbles, particles (e.g., aluminum), or wires. The inserts provide acoustic reflection for measuring temperature. The acoustic energy may propagate through the pad 30, but the inserts or pad itself may reflect at least some of the acoustic energy back to the transducer 54.

In one embodiment, the pad 30 is embedded with a small amount of acoustic scatterers. The pad 30 may be less efficient in passing the acoustic energy for therapy (e.g., in passing HIFU through the pad 30). The pad 30 may be less acoustically transparent as compared to no scatterers being embedded. The scatterers may cause a greater increase in temperature in the pad 30, but also allowing monitoring of temperature changes within the pad 30.

The pad 30 has any shape. The shape is a slab, such as a thin plate having a thickness of 1-3 cm. The length and width are sufficient to cover an aperture of the transducer 54. An indentation may be provided for the transducer 54. The surface of the pad 30 for contact with the patient may be curved. For example, different pads have different surface shapes for use with different patients and/or different locations on a patient.

The shape may be generally fixed, such as having a shape that may be compressed, distorting the shape, but returns upon release of the pressure. The shape may be malleable, such as associated with a liquid (e.g., water) filled pillow. Alternatively, the shape is fixed or relatively uncompressable.

The pad 30 is separate from the transducer 54. The user may select the pad 30 and position the pad 30 against the patient. Straps, medical glue, gel, or pressure may be used to hold the pad 30 against the patient.

In other embodiments, the pad 30 is integrated with the transducer 54. The pad 30 connects with the housing of the transducer such that movement of the transducer 54 moves the pad 30. The pad 30 is the point of contact with skin of the patient by the hand or robotic held therapy probe. The connection is by a strap, bolt, clip, or other mating. The pad 30 may be formed as part of the housing of the transducer 54, such as being part of a unitary construction with the housing.

The transmit beamformer 52 is one or more waveform generators, amplifiers, delays, phase rotators, multipliers, summers, digital-to-analog converters, filters, combinations thereof and other now known or later developed transmit beamformer components. The transmit beamformer 52 is configured into a plurality of channels for generating transmit signals for each element of a transmit aperture. The transmit signals for the elements are delayed and apodized relative to each other for focusing acoustic energy along one or more scan lines. Signals of different amplitudes, frequencies, bandwidths, delays, spectral energy distributions or other characteristics are generated for one or more elements during a transmit event.

For temperature measurement, the transmit beamformer 52 transmits one or more beams in a scan pattern. Upon transmission of acoustic waves from the transducer 54 in response to the generated waves, one or more beams are formed. A sequence of transmit beams are generated to scan a one, two or three-dimensional region. Sector, Vector®, linear, or other scan formats may be used. The same region is scanned multiple times. For strain imaging, scan or frame interleaving may be used (i.e., scan the entire region before scanning again). In alternative embodiments, the transmit beamformer 52 generates a plane wave or diverging wave for more rapid scanning.

For therapy, the transmit beamformer 52 transmits one or more beams. The transmit beamformer 52 causes generation of acoustic energy for HIFU. The therapy applicator (e.g., high intensity focused ultrasound transducer 54) generates high intensity focused ultrasound therapy waveforms. Relative delays focus the acoustic energy. A given transmit event corresponds to transmission of acoustic energy by different elements at a substantially same time given the delays. The transmit event provides a pulse of ultrasound energy for treating the tissue. The transmit event may be repeated and/or may include on-going (multiple cycle) waveforms.

The receive beamformer 56 is configured to acquire ultrasound data representing the pad 30. The ultrasound data is for measuring temperature. Other sources of data include sensors, a therapy system, or other inputs. Such inputs may be provided to the processor 62 or the memory 64.

The receive beamformer 56 includes a plurality of channels for separately processing signals received from different elements of the transducer 54. Each channel may include delays, phase rotators, amplifiers, filters, multipliers, summers, analog-to-digital converters, control processors, combinations thereof and other now known or later developed receive beamformer components. The receive beamformer 56 also includes one or more summers for combining signals from different channels into a beamformed signal. A subsequent filter may also be provided. Other now known or later developed receive beamformers may be used. Electrical signals representing the acoustic echoes from a transmit event are passed to the channels of the receive beamformer 56. The receiver beamformer outputs in-phase and quadrature, radio frequency or other data representing one or more locations in a scanned region. The channel data or receive beamformed data prior to detection may be used by the processor 62.

If imaging is provided, receive beamformed signals are subsequently detected and used to generate an ultrasound image by the image processor 58. The image processor 58 is a B-mode/M-mode detector, Doppler/flow/tissue motion estimator, harmonic detector, contrast agent detector, spectral Doppler estimator, combinations thereof, or other now known or later developed device for generating an image from received signals. The image processor 58 may include a scan converter. The detected or estimated signals, prior to or after scan conversion, may be used by the processor 62.

The display 60 is a monitor, LCD, plasma, projector, printer, or other now known or later developed display device. The display 60 is configured to display a skin temperature, therapy control information, and/or an image representing the effect of thermal therapy. For example, the skin temperature is output as a value, graph, or two-dimensional representation. The processor 62 and/or the image processor 58 generate display signals for the display 60. The display signals, such as RGB values, may be used by the processor 62.

The processor 62 is a control processor, beamformer processor, general processor, application specific integrated circuit, field programmable gate array, digital components, analog components, hardware circuit, combinations thereof and other now known or later developed devices for processing information. The processor 62 is configured, with computer code, to model an effect of thermal therapy on a treatment region and/or skin temperature. For example, the skin temperature for one or more locations is estimated based on inputs. The computer code implements a machine-learned model and/or other model to estimate the skin temperature. The model is a matrix, algorithm, or combinations thereof to estimate based on one or more input features.

The processor 62 controls generation of heat in the patient. The control is performed as a function of ultrasound data representing the pad. Other information may be used to control the therapy as well. For the ultrasound data from the pad, the ultrasound data is used for consideration of the skin temperature in the control of therapy. Any measure of the characteristics of the pad at a given time or change in the characteristic over time may be mapped to temperature of the pad and/or control of therapy based on temperature even without calculation of a specific temperature. Given a relationship between pad temperature and/or measured characteristic and the skin temperature, the skin temperature may be determined from the measurements. Based on the skin temperature (e.g., such as represented by the measurements of the pad and/or the pad temperature), the application of focused ultrasound from the transducer may be altered. If the temperature is too high, the acoustic energy may be reduced or turned off, at least for a cool down period. If the temperature is low, the acoustic energy may be increased. The pattern or aperture used to apply the acoustic energy may be shifted or changed based on the skin temperature. Spatial and/or temporal distribution of skin temperature may be used to control application.

The processor 62 inputs the measurements from the pad, pad temperature and/or other information into a model to determine the skin temperature and/or control due to skin temperature. The model may be a look-up table or programmed function, such as associated with experimentally determined relationship of skin temperature to pad measurements. In other embodiments, the model is a matrix or other representation (e.g., coefficients) of a machine-trained model. The model outputs a skin temperature and/or control based on skin temperature. The processor 62 may implement a dose model and/or thermal distribution model.

The memory 64 is a non-transitory computer readable storage medium having stored therein data representing instructions executable by the programmed processor for determining skin temperature in medical ultrasound therapy. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method of determining skin temperature in medical ultrasound therapy, the method comprising:
positioning a standoff between a therapy transducer and skin of a patient;
applying a thermal dose from the therapy transducer, through the standoff, through the skin, and into the patient, the thermal dose focused at a region in the patient such that the region is heated in response to the thermal dose;
acquiring, with the therapy transducer, ultrasound data representing acoustic echoes from the standoff adjacent to the skin;
determining the skin temperature as a function of the acoustic echoes; and
controlling the applying as a function of the skin temperature.

2. The method of claim 1 wherein positioning comprises positioning the therapy transducer about two centimeters or less from the skin of the patient.

3. The method of claim 1 wherein positioning comprises positioning first gel between the skin and the standoff and positioning second gel between the therapy transducer and the standoff.

4. The method of claim 1 wherein applying comprises applying high intensity focused ultrasound from the therapy transducer, the therapy transducer comprising an array of elements.

5. The method of claim 1 wherein applying comprises generating acoustic energy with the therapy transducer, the acoustic energy propagating from the therapy transducer to the region.

6. The method of claim 1 wherein acquiring comprises transmitting to locations at an interface of the standoff with the skin and receiving the acoustic echoes from the locations.

7. The method of claim 1 wherein acquiring comprises receiving the acoustic echoes from scatterers in the standoff.

8. The method of claim 1 wherein determining comprises measuring displacement, speed of sound, backscatter intensity, and a normalized correlation coefficient of received signals.

9. The method of claim 1 wherein determining comprises applying received signals representing the acoustic echoes to a machine-trained model.

10. The method of claim 1 wherein controlling comprises terminating the applying or reducing the thermal dose where the skin temperature is above a threshold.

11. The method of claim 1 wherein determining the skin temperature comprises estimating the skin temperature from a measured characteristic of the standoff.

12. The method of claim 11 wherein estimating comprises estimating with a thermal distribution model.

13. The method of claim 1 wherein controlling comprises inputting the skin temperature to a dosimetry model.

14. In a non-transitory computer readable storage medium having stored therein data representing instructions executable by a programmed processor for determining skin temperature in medical ultrasound therapy, the storage medium comprising instructions for:
performing a high intensity focused ultrasound (HIFU) procedure with a HIFU applicator;
monitoring a phantom temperature of a phantom material between the HIFU applicator and a patient;
relating the phantom temperature to a skin temperature; and
adjusting the HIFU procedure based on the skin temperature.

15. The non-transitory computer readable storage medium of claim 14 wherein monitoring comprises receiving signals reflected from the phantom material with the HIFU applicator.

16. The non-transitory computer readable storage medium of claim 14 wherein relating comprises identifying the skin temperature based on thermal strain of the phantom material adjacent to an interface of the phantom material with skin of the patient.

17. The non-transitory computer readable storage medium of claim 14 wherein adjusting comprises ceasing the performing of the HIFU procedure or altering an acoustic energy of the HIFU procedure.

18. A system for controlling skin temperature in medical ultrasound therapy, the system comprising:
an array of transducer elements;
a pad operable to allow propagation of acoustic energy from the array and reflect the acoustic energy to the array;
a receive beamformer configured to acquire ultrasound data representing the pad; and
a processor configured to control generating of heat in a patient as a function of the ultrasound data representing the pad.

19. The system of claim 18 wherein the pad connects with the array such that movement of the array moves the pad, the pad being a point of contact with skin of the patient.

20. The system of claim 18 wherein the processor is configured to determine a temperature of the pad from the ultrasound data, determine a skin temperature from the temperature of the pad, and alter application of focused ultrasound from the array based on the skin temperature.
